(19) United States
(12) Patent Application Publication
(10) Pub. No.: US 2024/0050486 A1
(43) Pub. Date: Feb. 15, 2024

(54) METHODS OF GENERATING CORTICAL EXCITATORY NEURONS

(71) Applicants: Memorial Sloan-Kettering Cancer Center, New York, NY (US); Sloan-Kettering Institute For Cancer Research, New York, NY (US); Memorial Hospital for Cancer and Allied Disease, New York, NY (US)

(72) Inventors: Lorenz Studer, New York, NY (US); Gabriele CICERI, New York, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 18/348,399

(22) Filed: Jul. 7, 2023

Related U.S. Application Data
(63) Continuation of application No. PCT/US2022/011539, filed on Jan. 7, 2022.
(60) Provisional application No. 63/134,651, filed on Jan. 7, 2021.

(51) Int. Cl. A61K 35/30; C12N 5/0793; A61P 25/28

(52) U.S. Cl. CPC A61K 35/30; C12N 5/0619; A61P 25/28; C12N 2506/03; C12N 2501/415; C12N 2501/13; C12N 2533/90; C12N 2501/42

(57) ABSTRACT

The present disclosure provides methods for generating cortical excitatory neurons, cortical excitatory neurons generated by such methods, and composition comprising such cells. The present disclosure also provides uses of the cortical excitatory neurons and composition comprising thereof for preventing and/or treating neurodegenerative disorders.

### CROSS-REFERENCE TO RELATED APPLICATIONS

This application is a Continuation application of International Patent Application No. PCT/US2022/011539, filed on Jan. 7, 2022, which claims priority to U.S. Provisional Application No. 63/134,651 filed Jan. 7, 2021, the content of which is incorporated by reference in its entirety herein, and to which priority is claimed.

### 1. INTRODUCTION

The present disclosure provides methods for generating cortical excitatory neurons, cortical excitatory neurons generated by such methods, and compositions comprising such cortical excitatory neurons. The present disclosure also provides uses of the cortical excitatory neurons and compositions comprising thereof for preventing and/or treating neurodegenerative disorders, neurodevelopmental disorders, and/or neuropsychiatric disorders.

### 2. BACKGROUND

Previously, embryonic and somatic stem cells were used as therapeutics and model systems for neurodegenerative diseases, neurodevelopmental disorders, and/or neuropsychiatric disorders. Research and technological developments relating to directed differentiation of embryonic and somatic stem cells has taken place in the field of diseases of the central nervous system (CNS), such as for Alzheimer's disease, Frontotemporal dementia, Parkinson's disease, schizophrenia, Autism, Depression, Intellectual disabilities, Amyotrophic lateral sclerosis, and Stroke. However, the results of these studies showed little in vivo capability to restore neuronal function and often resulted in the growth of unwanted tumors in the patients.

Therefore, there is still a need for improved methods for generating cortical neurons that are suitable for treating neurodegenerative disorders, neurodevelopmental disorders, and/or neuropsychiatric disorders.

### 3. SUMMARY OF THE INVENTION

The present disclosure provides methods for generating cortical excitatory neurons, cortical excitatory neurons generated by such methods, and compositions comprising such cells. The present disclosure also provides uses of the cortical excitatory neurons and compositions comprising thereof for preventing and/or treating neurodegenerative disorders, neurodevelopmental disorders, and/or neuropsychiatric disorders.

In certain embodiments, the present disclosure provides an in vitro method for inducing differentiation of stem cells, comprising An in vitro method for inducing differentiation of stem cells, comprising: contacting the stem cells with at least one inhibitor of Small Mothers Against Decapentaplegic (SMAD) signaling, and at least one inhibitor of wingless (Wnt) signaling; and contacting the cells with at least one inhibitor of Notch signaling to obtain a cell population of differentiated cells, wherein at least about 50% of the differentiated cells express at least one cortical excitatory neuron marker; wherein the initial contact of the cells with the at least one inhibitor of Notch signaling is at least about 10 days from the initial contact of the cells with the at least one inhibitor of SMAD signaling. In certain embodiments, at least about 75% of the differentiated cells express the at least one cortical excitatory neuron marker. In certain embodiments, at least about 95% of the differentiated cells express the at least one cortical excitatory neuron marker.

In certain embodiments, the initial contact of the cells with the at least one inhibitor of Notch signaling is about 20 days from the initial contact of the cells with the at least one inhibitor of SMAD signaling.

In certain embodiments, the cells are contacted with the at least one inhibitor of Notch signaling for at least about 1 day.

In certain embodiments, the cells are contacted with the at least one inhibitor of Notch signaling for up to about 20 days.

In certain embodiments, the cells are contacted with the at least one inhibitor of Notch signaling for about 10 days.

In certain embodiments, the cells are contacted with the at least one inhibitor of SMAD signaling for about 10 days.

In certain embodiments, the cells are contacted with the at least one inhibitor of Wnt signaling for up to about 3 days.

In certain embodiments, the at least one inhibitor of Notch signaling comprises a γ-secretase inhibitor.

In certain embodiments, the 7-secretase inhibitor comprises DAPT, derivatives thereof, or mixtures thereof.

In certain embodiments, the at least one inhibitor of SMAD signaling is selected from inhibitors of TGFβ/Activin-Nodal signaling, inhibitors of bone morphogenetic protein (BMP) signaling, and combinations thereof.

In certain embodiments, the at least one inhibitor of TGFβ/Activin-Nodal signaling comprises an inhibitor of ALK5.

In certain embodiments, the at least one inhibitor of TGFβ/Activin-Nodal signaling comprises SB431542, or a derivative, or a mixture thereof.

In certain embodiments, the at least one inhibitor of TGFβ/Activin-Nodal signaling comprises SB431542.

In certain embodiments, the at least one inhibitor of BMP signaling comprises LDN193189, Noggin, dorsomorphin, a derivative thereof, or a mixture thereof.

In certain embodiments, the at least one inhibitor of BMP comprises LDN-193189.

In certain embodiments, the at least one inhibitor of Wnt signaling comprises a compound selected from the group consisting of XAV939, IWP2, DKK1, IWR1, IWP L6, Wnt-059, JW 55, derivatives thereof, and combinations thereof.

In certain embodiments, the at least one inhibitor of Wnt signaling comprises XAV939.

In certain embodiments, the at least one cortical excitatory neuron marker is selected from TBR1 (T-Box Brain 1), MAP2 (Microtubule-Associated Protein 2), FOXG1, CTIP2, DCX, TUBB3, FOXP2, vGlut1/2, TLE4, and combinations thereof.

In certain embodiments, the differentiated cells do not express at least one marker selected from KI67, CTIP2 (Chicken Ovalbumin Upstream Promoter Transcription Factor Interacting Protein 2), SATB2 (Special AT-Rich Sequence-Binding Protein 2), and combinations thereof.

In certain embodiments, the stem cells are selected from human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells.

In certain embodiments, the stem cells are human stem cells.

In certain embodiments, the stem cells are pluripotent or multipotent stem cells.

In certain embodiments, the stem cells are pluripotent stem cells.

In certain embodiments, the pluripotent stem cells are selected from embryonic stem cells, induced pluripotent stem cells, and combinations thereof.

In certain embodiments, the present disclosure provides a cell population of in vitro differentiated cells, wherein said in vitro differentiated cells are obtained by a presently disclosed method.

In certain embodiments, the present disclosure provides a cell population of in vitro differentiated cells, wherein at least about 50%, at least about 75%, or at least about 95% of the cells express at least one cortical excitatory neuron marker.

In certain embodiments, the at least one cortical excitatory neuron marker is selected from TBR1, MAP2, CTIP2, FOXG1, DCX, TUBB3, FOXP2, vGlut1/2, TLE4, and combinations thereof.

In certain embodiments, less than about 30% of the differentiated cells express at least one marker selected from KI67, CTIP2, SATB2, and combinations thereof.

In certain embodiments, the present disclosure provides a composition comprising the presently disclosed cell population.

In certain embodiments, the composition a pharmaceutical composition further comprising a pharmaceutically acceptable carrier.

In certain embodiments, the present disclosure provides a kit for inducing differentiation of stem cells to cortical excitatory neurons, comprising:
(a) at least one inhibitor of SMAD signaling;
(b) at least one inhibitor of Wnt signaling; and
(c) at least one inhibitor of Notch signaling.

In certain embodiments, the kit further comprises (d) instructions for inducing differentiation of the stem cells into a population of differentiated cells expressing at least one cortical excitatory neuron marker.

In certain embodiments, the present disclosure provides a method of preventing and/or treating a neurodegenerative disorder, a neurodevelopmental disorder, and/or a neuropsychiatric disorder in a subject, comprising administering to the subject an effective amount of the cell population disclosed herein or the composition disclosed herein.

In certain embodiments, the neurodegenerative disorder, or neurodevelopmental disorder, and/or neuropsychiatric disorder is selected from Alzheimer's disease, Frontotemporal dementia, Parkinson's disease, schizophrenia, Autism, Depression, Intellectual disabilities, Amyotrophic lateral sclerosis, and Stroke.

In certain embodiments, the present disclosure provides the presently disclosed cell population or the presently disclosed composition for use in preventing and/or treating a neurodegenerative disorder, a neurodevelopmental disorder, and/or a neuropsychiatric disorder in a subject.

In certain embodiments, the neurodegenerative disorder, neurodevelopmental disorder, and/or neuropsychiatric disorder is selected from Alzheimer's disease, Frontotemporal dementia, Parkinson's disease, schizophrenia, Autism, Depression, Intellectual disabilities, Amyotrophic lateral sclerosis, and Stroke.

### 4. BRIEF DESCRIPTION OF THE DRAWINGS

FIGS. 1A-1C show robust induction of cortical progenitor cells from hPSCs. FIG. 1A depicts methods of generating cortical excitatory neurons in accordance with certain embodiments of the presently disclosed subject matter. FIG. 1B shows qRT-PCR analysis data, which showed progressive and robust induction of telencephalic cortical markers and efficient downregulation of pluripotency associated genes. FIG. 1C depicts homogeneous induction of cortical specific genes FOXG1 and PAX6 and minimal contamination of ventral telencephalic markers.

FIGS. 2A-2D show rapid depletion of progenitor cells and synchronized neurogenesis. FIG. 2A shows that Ki67⁺ progenitor cells were rapidly depleted and a pure population of MAP2⁺ neurons were generated by day 25 of differentiation. FIG. 2B provides EdU birthdating studies, which confirmed the generation of roughly isochronic neuron. FIG. 2C shows the quantification. FIG. 2D shows generation of a highly pure population of neurons having a TBR1+identity (>80%) by synchronized neurogenesis.

FIGS. 3A-3B show maintenance of synchronized and pure hPSCs-derived cortical neurons at high viability in long-term cultures. FIG. 3A provides representative images of hPSCs-derived cortical neurons in long-term culture. FIG. 3B provides qRT-PCR analyses for KI67/MAP2 marker expression, which confirmed the maintenance of synchronicity and purity conditions in long-term cultures.

### 5. DETAILED DESCRIPTION

The present disclosure provides methods for generating cortical excitatory neurons, cortical excitatory neurons generated by such methods, and compositions comprising such cells. The present disclosure also provides uses of the cortical excitatory neurons and compositions comprising thereof for preventing and/or treating neurodegenerative disorders.

The present disclosure is at least based on the discovery that the presently disclosed methods generated a population of synchronized cortical excitatory neurons with a high purity. Moreover, the generated cortical excitatory neurons can be maintained in vivo at high viability and purity long-term.

Non-limiting embodiments of the presently disclosed subject matter are described by the present specification and Examples.

For purposes of clarity of disclosure and not by way of limitation, the detailed description is divided into the following subsections:
5.1. Definitions;
5.2. Methods of Differentiating Stem Cells;
5.3. Cell Populations and Compositions;
5.4. Methods of Treating Neurodegenerative Disorders; and
5.5. Kits.

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, and a wingless (Wnt) complex protein, including beta-catenin, NOTCH, transforming growth factor beta (TGFP), Activin, Nodal, and glycogen synthase kinase 3β (GSK3β) proteins, and bone morphogenetic proteins (BMP). For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGP), Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, a named associated molecule, such as a glycogen synthase kinase 3β (GSK3β)) (e.g., including, but not limited to, the signaling molecules described herein), for one example, via directly contacting SMAD signaling, contacting SMAD mRNA, causing conformational changes of SMAD, decreasing SMAD protein levels, or interfering with SMAD interactions with signaling partners (e.g., including those described herein), and affecting the expression of SMAD target genes (e.g. those described herein).

Inhibitors also include molecules that indirectly regulate biological activity, for example, SMAD biological activity, by intercepting upstream signaling molecules (e.g., within the extracellular domain, examples of a signaling molecule and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules). Antibodies that block upstream or downstream proteins are contemplated for use to neutralize extracellular activators of protein signaling, and the like. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

As used herein, the term “WNT” or “wingless” in reference to a ligand refers to a group of secreted proteins (e.g., integration 1 in humans) that are capable of interacting with a WNT receptor, such as a receptor in the Frizzled and LRPDerailed/RYK receptor family. As used herein, the term “a WNT or wingless signaling pathway refers to a signaling pathway composed of Wnt family ligands and Wnt family receptors, such as Frizzled and LRPDerailed/RYK receptors, mediated with or without β-catenin. In certain embodiments, the WNT signaling pathway include mediation by β-catenin, e.g., WNT/-catenin.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of cortical excitatory neurons, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation.

As used herein, the term “neuron” refers to a nerve cell, the principal functional units of the nervous system. A neuron consists of a cell body and its processes—an axon and at least one dendrite. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as neural, neural crest, cranial placode, and non-neural ectoderm precursors. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker of cortical excitatory neurons, such as TBR1, MAP2, CTIP2, FOXG1, DCX, TUBB3, FOXP2, vGlut1/2, and TLE4).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “cortical excitatory neuron” refers to a type of cerebral cortex neurons that can release a neurotransmitter, such as glutamate, that can result in a connected post-synaptic neuron to become more likely to fire. “Cortical excitatory neuron” also refers to “cortical projection neuron”. Non-limiting examples of cortical excitatory neurons include corticofugal projection neurons and cortico-cortical projection neurons.

### 5.2. Method of Differentiating Stem Cells

The present disclosure provides for in vitro methods for inducing differentiation of stem cells (e.g., human stem cells). The presently disclosed subject matter provides in vitro methods for inducing differentiation of stem cells to produce cortical neurons, e.g., cortical excitatory neurons. In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include human, nonhuman primate or rodent nonembryonic stem cells, embryonic stem cells, induced nonembryonic pluripotent cells and engineered pluripotent cells. In certain embodiments, the stem cells are human stem cells. Non-limiting examples of human stem cells include human pluripotent stem cell (hPSC) (including, but not limited to human embryonic stem cells (hESC) and human induced pluripotent stem cells (hiPSC)), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell is an embryonic stem cell (ESC). In certain embodiments, the stem cell is a human embryonic stem cell (hESC). In certain embodiments, the stem cell is an induced pluripotent stem cell (iPSC). In certain embodiments, the stem cell is a human induced pluripotent stem cell (hiPSC).

The present disclosure is directed to stem cell-derived cortical neurons, e.g., cortical excitatory neurons. In certain embodiments, the differentiation of stem cells to cortical excitatory neurons includes in vitro differentiation of stem cells to cells expressing at least one cortical progenitor marker (also referred to as “cortical progenitors”), and in vitro differentiation of cells expressing at least one cortical progenitor marker to cells expressing at least one cortical excitatory neuron marker (also referred to as “cortical excitatory neurons”).

In certain embodiments, the present disclosure provides methods for inducing differentiation of stem cells, comprising contacting stem cells with at least one inhibitor of Small Mothers Against Decapentaplegic (SMAD) signaling (referred to as “SMAD inhibitor”), and at least one inhibitor of wingless (Wnt) signaling (referred to as “Wnt inhibitor”); and contacting the cells with at least one inhibitor of Notch signaling (referred to as “Notch inhibitor”), to obtain a cell population comprising differentiated cells expressing at least one cortical excitatory neuron marker.

### 5.2.1. Differentiation of Stem Cells to Cortical Progenitors

In certain embodiments, the cells expressing at least one cortical progenitor marker are in vitro differentiated from stems cells by contacting stem cells (e.g., human stem cells) with at least one inhibitor of SMAD signaling, and at least one inhibitor of Wnt signaling.

Non-limiting examples of cortical progenitor markers include FOXG1 (Forkhead Box G1), PAX6 (Paired Box 6), EMX2 (Empty Spiracles Homeobox 2), FEZF2 (FEZ Family Zinc Finger 2), OTX2, NEUROG1, NEUROG2, HESS, and combinations thereof.

Non-limiting examples of SMAD inhibitors include inhibitors of transforming growth factor beta (TGFβ)/Activin-Nodal signaling (referred to as “TGFP/Activin-Nodal inhibitor”), and inhibitors of bone morphogenetic proteins (BMP) signaling. In certain embodiments, the TGFβ/Activin-Nodal inhibitor can neutralize the ligands including TGFβs, BMPs, Nodal, and activins, and/or block their signal pathways through blocking the receptors and downstream effectors. Non-limiting examples of TGFβ/Activin-Nodal inhibitors include those disclosed in WO/2010/096496, WO/2011/149762, WO/2013/067362, WO/2014/176606, WO/2015/077648, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20 (2012), all of which are incorporated by reference in their entireties herein for all purposes. In certain embodiments, the at least one TGFβ/Activin-Nodal inhibitor is selected from inhibitors of ALK5, inhibitors of ALK4, inhibitors of ALK7, and combinations thereof). In certain embodiments, the TGFβ/Activin-Nodal inhibitor comprises an inhibitor of ALK5. In certain embodiments, the TGFP/Activin-Nodal inhibitor is a small molecule selected from SB431542, derivatives thereof; and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

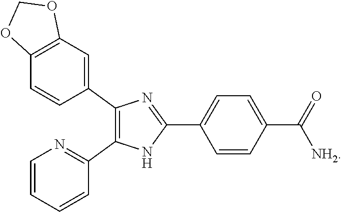 [SMILES: NC(=O)c1ccc(-c2nc(-c3ccc4c(c3)OCO4)c(-c3ccccn3)[nH]2)cc1]

In certain embodiments, the TGFP/Activin-Nodal inhibitor comprises SB431542. In certain embodiments, the TGFP/Activin-Nodal inhibitor comprises a derivative of SB431542. In certain embodiments, the derivative of SB431542 is A83-01.

In certain embodiments, the at least one SMAD inhibitor comprises an inhibitor of BMP signaling (referred to as “BMP inhibitor”). Non-limiting examples of BMP inhibitors include those disclosed in WO2011/149762, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20, all of which are incorporated by reference in their entireties. In certain embodiments, the BMP inhibitor is a small molecule selected from LDN193189, Noggin, dorsomorphin, derivatives thereof; and mixtures thereof. “LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆ with the following formula.

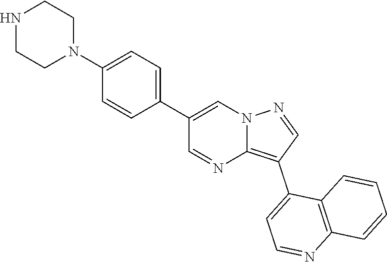 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18: 4388-4392, herein incorporated by reference).

In certain embodiments, the BMP inhibitor comprises LDN193189. In certain embodiments, the BMP inhibitor comprises Noggin.

In certain embodiments, the stem cells are exposed to one SMAD inhibitor, e.g., one TGFβ/Activin-Nodal inhibitor. In certain embodiments, the one TGFβ/Activin-Nodal inhibitor is SB431542 or A83-01. In certain embodiments, the stem cells are exposed to two SMAD inhibitors. In certain embodiments, the two SMAD inhibitors are a TGFβ/Activin-Nodal inhibitor and a BMP inhibitor. In certain embodiments, the stem cells are exposed to SB431542 or A83-01, and LDN193189 or Noggin. In certain embodiments, the stem cells are exposed to SB431542 and LDN193189.

In certain embodiments, the stem cells are exposed to or contacted with at least one SMAD inhibitor for at least about 3 days, at least about 5 days, at least about 10 days, or at least about 15 days. In certain embodiments, the stem cells are exposed to or contacted with at least one SMAD inhibitor for at least about 3 days. In certain embodiments, the stem cells are contacted with or exposed to the at least one SMAD inhibitor for up to about 5 days, up to about 10 days, up to about 15 days, or up to about 20 days. In certain embodiments, the stem cells are contacted with or exposed to the at least one SMAD inhibitor for up to about 20 days. In certain embodiments, the stem cells are contacted with or exposed to the at least one SMAD inhibitor for between about 3 days and about 20 days, between about 3 days and about 15 days, between about 3 days and about 10 days, between about 10 days and about 20 days, or between about 10 days and about 15 days. In certain embodiments, the stem cells are contacted with or exposed to the at least one SMAD inhibitor between about 3 days and about 15 days. In certain embodiments, the stem cells are contacted with or exposed to the at least one SMAD inhibitor for about 5 days, about 10 days, about 15 days, or about 20 days. In certain embodiments, the stem cells are contacted with or exposed to the at least one SMAD inhibitor for about 10 days. In certain embodiments, the stem cells are contacted with or exposed to the at least one SMAD inhibitor for 9 days or 10 days. In certain embodiments, the stem cells are contacted with or exposed to the at least one SMAD inhibitor from day 0 through day 9. In certain embodiments, the at least one SMAD inhibitor is added every day or every other day to a cell culture medium comprising the stem cells from day 0 through day 9. In certain embodiments, the at least one SMAD inhibitor is added every day (daily) to a cell culture medium comprising the stem cells from day 0 to day 9.

In certain embodiments, the at least one SMAD inhibitor comprises a TGFβ/Activin-Nodal inhibitor. the cells are contacted with or exposed to a TGFβ/Activin-Nodal inhibitor. In certain embodiments, the concentration of the TGFβ/Activin-Nodal inhibitor contacted with or exposed to the cells is between about 1 μM and about 20 μM, between about 1 μM and about 10 μM, between about 1 μM and about 15 μM, between about 10 μM and about 15 μM, between about 5 μM and about 10 μM, between about 5 μM and about 15 μM, between about 5 μM and about 20 μM, or between about 15 μM and about 20 μM. In certain embodiments, the concentration of the TGFβ/Activin-Nodal inhibitor contacted with or exposed to the cells is between about 5 μM and about 10 μM. In certain embodiments, the concentration of the TGFβ/Activin-Nodal inhibitor contacted with or exposed to the cells is about 10 μM. In certain embodiments, the TGFβ/Activin-Nodal inhibitor comprises SB431542 or a derivative thereof (e.g., A83-01). In certain embodiments, the TGFβ/Activin-Nodal inhibitor comprises SB431542.

In certain embodiments, the at least one SMAD inhibitor comprises a BMP inhibitor. In certain embodiments, the cells are contacted with or exposed to a BMP inhibitor. In certain embodiments, the concentration of the BMP inhibitor contacted with or exposed to the cells is between about 10 nM and about 200 nM, or between about 10 nM and about 50 nM, or between about 10 nM and about 150 nM, or between about 10 nM and about 100 nM, or between about 50 nM and about 200 nM, or between about 50 nM and about 150 nM, or between about 50 nM and about 100 nM, or between about 100 nM and about 200 nM, or between about 100 nM and about 150 nM. In certain embodiments, the concentration of the BMP inhibitor contacted with or exposed to the cells is between about 50 nM and about 150 nM. In certain embodiments, the concentration of the BMP inhibitor contacted with or exposed to the cells is between about 50 nM and about 100 nM. In certain embodiments, the concentration of the BMP inhibitor contacted with or exposed to the cells is about 100 nM. In certain embodiments, the BMP inhibitor comprises LDN193189 or a derivative thereof. In certain embodiments, the BMP inhibitor comprises LDN193189.

In certain embodiments, the at least one SMAD inhibitor comprises a TGFβ/Activin-Nodal inhibitor and a BMP inhibitor. In certain embodiments, the cells are contacted with or exposed to the TGFβ/Activin-Nodal inhibitor and the BMP inhibitor simultaneously. In certain embodiments, the stem cells are contacted with or exposed to the TGFβ/Activin-Nodal inhibitor and the BMP inhibitor for about 10 days. In certain embodiments, the stem cells are contacted with or exposed to the TGFβ/Activin-Nodal inhibitor and the BMP inhibitor for 9 days or 10 days. In certain embodiments, the cells are contacted with or exposed to the TGFβ/Activin-Nodal inhibitor and the BMP inhibitor from day 0 through day 9. In certain embodiments, the TGFβ/Activin-Nodal inhibitor and the BMP inhibitor are exposed to or contacted with the cells daily (e.g., every day) or every other day. In certain embodiments, the TGFβ/Activin-Nodal inhibitor and the BMP inhibitor are added every day or every other day to a cell culture medium comprising the stem cells from day 0 through day 9. In certain embodiments, the TGFβ/Activin-Nodal inhibitor and the BMP inhibitor are added every day (daily) to a cell culture medium comprising the stem cells from day 0 to day 9.

“Wingless” or “Wnt” refers to a signal pathway composed of Wnt family ligands and Wnt family receptors, such as Frizzled and LRPDerailed/RYK receptors, mediated with or without β-catenin. Wnt proteins have been implicated in oncogenesis and in several developmental processes, including regulation of cell fate and patterning during embryogenesis. The Wnt pathway includes any of the proteins downstream or upstream of Wnt protein activity. For example, this could include LRPS, LRP6, Dkk, GSK-3, Wnt10B, Wnt6, Wnt3 (e.g., Wnt 3A), Wnt1 or any of the other proteins discussed herein, and the genes that encode these proteins.

The Wnt pathway also includes pathways that are downstream of Wnt, such as the LRPS or HBM pathways, the Dkk pathway, the p-catenin pathway, the MAPKAPK2 pathway, the OPG/RANK pathway, and the like. By “LRPS pathway” and “IBM pathway” is meant any proteins/genes including LRPS or the HBM mutant and proteins downstream of LRPS or the HBM mutant. By “β-catenin pathway” is meant any proteins/genes including β-catenin and proteins downstream of β-catenin. By “MAPKAPK2 pathway” is meant any proteins/genes including MAPKAPK2 and proteins downstream of MAPKAPK2. By “OPG/RANKL pathway” is meant any proteins/genes including OPG/RANKL and proteins downstream of OPG and RANKL. By “Dkk pathway” is meant to include any proteins/genes involved in Dkk-1 and LRPS and/or LRP6 interaction that is part of the Wnt pathway. Dkk-I inhibits LRPS activity.

The term “inhibitor of Wnt signaling” or “Wnt inhibitor” as used herein refers not only to any agent that may act by directly inhibiting the normal function of the Wnt protein, but also to any agent that inhibits the Wnt signaling pathway, and thus recapitulates the function of Wnt. Examples of the Wnt inhibitors include XAV939 (Hauang et al. Nature 461:614-620 (2009)), vitamin A (retinoic acid), lithium, flavonoid, Dickkopf1 (Dkk1), insulin-like growth factor-binding protein (IGFBP) (WO2009/131166), and siRNAs against β-catenin.

Non-limiting examples of inhibitor of Wnt signaling include XAV939, IWP2, DKK1, IWR1, IWP L6, Wnt-059, JW 55, derivatives thereof, and combinations thereof.

Additional Wnt inhibitors include, but are not limited to, IWR compounds, IWP compounds, and other Wnt inhibitors described in WO09155001 and Chen et al., Nat Chem Biol 5:100-7 (2009).

XAV939 is a potent, small molecule inhibitor of tankyrase (TNKS) 1 and 2 with IC₅₀ values of 11 and 4 nM, respectively. Huang et al., Nature 461:614-620 (2009). By inhibiting TNKS activity, XAV939 increases the protein levels of the axin-GSK3β complex and promotes the degradation of β-catenin in SW480 cells. Known Wnt inhibitors also include Dickkopf proteins, secreted Frizzled-related proteins (sFRP), Wnt Inhibitory Factor 1 (WIF-1), and Soggy. Members of the Dickkopf-related protein family (Dkk-1 to -4) are secreted proteins with two cysteine-rich domains, separated by a linker region. Dkk-3 and -4 also have one prokineticin domain. Dkk-1, -2, -3, and -4 function as antagonists of canonical Wnt signaling by binding to LRP5/6, preventing LRP5/6 interaction with Wnt-Frizzled complexes. Dkk-1, -2, -3, and -4 also bind cell surface Kremen-1 or -2 and promote the internalization of LRP5/6. Antagonistic activity of Dkk-3 has not been demonstrated. Dkk proteins have distinct patterns of expression in adult and embryonic tissues and have a wide range of effects on tissue development and morphogenesis.

The Dkk family also includes Soggy, which is homologous to Dkk-3 but not to the other family members. The sFRPs are a family of five Wnt-binding glycoproteins that resemble the membrane-bound Frizzleds. The largest family of Wnt inhibitors, they contain two groups, the first consisting of sFRP1, 2, and 5, and the second including sFRP3 and 4. All are secreted and derived from unique genes, none are alternate splice forms of the Frizzled family. Each sFRP contains an N-terminal cysteine-rich domain (CRO). Other Wnt inhibitors include WIF-1 (Wnt Inhibitory Factor 1), a secreted protein that binds to Wnt proteins and inhibits their activity.

“IWP2” or “Inhibitor of WNT Production-2” refers to IUPAC name N-(6-methyl-1,3-benzothiazol-2-yl)-2-[(4-oxo-3-phenyl-6,7-dihydrothieno[3,2-d]pyrimidin-2-yl)thio]acetamide” with the following formula:

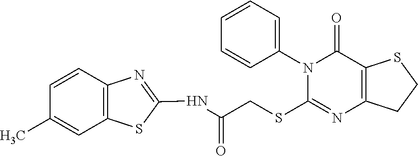 [SMILES: Cc1ccc2nc(NC(=O)CSc3nc4c(c(=O)n3-c3ccccc3)SCC4)sc2c1]

IWP-2 inhibits the WNT pathway (IC₅₀=27 nM) at the level of the pathway activator Porcupine. Porcupine is a membrane-bound acyltransferase that palmitoylates WNT proteins, which leads to WNT secretion and signaling capability.

In certain embodiments, the stem cells are contacted with or exposed to the at least one Wnt inhibitor for at least about 1 day, at least about 2 days, at least about 3 days, at least about 4 days, or at least about 5 days. In certain embodiments, the stem cells are contacted with the at least one Wnt inhibitor for up to about 1 day, up to about 5 days or up to about 10 days. In certain embodiments, the stem cells are contacted with the at least one Wnt inhibitor for up to about 5 days. In certain embodiments, the stem cells are contacted with the at least one Wnt inhibitor for up to about 3 days. In certain embodiments, the stem cells are contacted with the at least one Wnt inhibitor for between about 1 days and about 5 days. In certain embodiments, the stem cells are contacted with the at least one Wnt inhibitor for about 2 days. In certain embodiments, the stem cells are contacted with the at least one Wnt inhibitor for about 3 days. In certain embodiments, the stem cells are exposed to or contacted with the at least one Wnt inhibitor from day 0 through day 2. In certain embodiments, the stem cells are exposed to or contacted with the at least one Wnt inhibitor every day (daily) or every other day. In certain embodiments, the at least one Wnt inhibitor is added every day or every other day to a cell culture medium comprising the stem cells from day 0 through day 2. In certain embodiments, the at least one Wnt inhibitor is added every day (daily) to a cell culture medium comprising the stem cells from day 0 through day 2.

In certain embodiments, the concentration of the at least one Wnt inhibitor contacted with or exposed to the cells is from about 1 μM to about 20 μM, from about 1 μM to about 15 μM, from about 1 μM to about 10 μM, from about 1 μM to about 5 μM, from about 5 μM to about 10 μM, from about 5 μM to about 15 μM, from about 15 μM to about 20 μM, from about 5 μM to about 20 μM, or from about 10 μM to about 15 μM. In certain embodiments, the concentration of the at least one Wnt inhibitor contacted with or exposed to the cells is from about 1 μM to about 504. In certain embodiments, the cells are contacted with the at least one Wnt inhibitor in a concentration of about 2 μM. In certain embodiments, the Wnt inhibitor comprises XAV939 or a derivative thereof. In certain embodiments, the Wnt inhibitor comprises XAV939.

### 5.2.2. Differentiation of Cortical Progenitors to Cortical Excitatory Neurons

In certain embodiments, the cells expressing at least one cortical excitatory neuron marker are in vitro differentiated from cells expressing at least one cortical progenitor marker (e.g., those obtained by the method described in Section 5.2.1) by contacting cells expressing at least one cortical progenitor marker (e.g., those obtained by the method described in Section 5.2.1) with at least one inhibitor of Notch signaling (referred to as “Notch inhibitor”). The exposure of the cells to the at least one Notch inhibitor can promote rapid cell cycle exit and induce synchronized neurogenesis. In certain embodiments, the cells are passaged at low density to obtain synchronized and/or pure cortical excitatory neurons.

Non-limiting examples of cortical excitatory neuron markers include TBR1 (T-Box Brain 1), MAP2 (Microtubule-Associated Protein 2), FOXG1, CTIP2, DCX, TUBB3, FOXP2, vGlut1/2, and TLE4.

Non-limiting examples of Notch inhibitors include DAPT (Dovey et al., Journal of neurochemistry 76, 173-181 (2001)), Begacestat (5-Chloro-N-[(1S)-3,3,3-trifluoro-1-(hydroxymethyl)-2-(trifluoromethyl)propyl]-2-thiophenesulfonamide) (Mayer et al., J. Med. Chem. 51:7348 (2008)), DBZ (N-[(1S)-2-[[(7S)-6,7-Dihydro-5-methyl-6-oxo-5H-dibenz[b,d]azepin-7-yl]amino]-1-methyl-2-oxoethyl]-3,5-difluorobenzeneacetamide) (van Es et al., Nature 435:959 (2005)), BMS 299897 (2-[(1R)-1-[[(4-Chlorophenyl)sulfonyl](2,5-difluorophenyl)amino]ethyl-5-fluorobenzenebutanoic acid] (Goldstein et al., J. Pharmacol. Exp. Ther. 323:102 (2007)), Compound W (3,5-Bis(4-nitrophenoxy)benzoic acid) (Okochi et al., J. Biol. Chem. 281:7890 (2006)), Flurizan ((R)-2-Fluoro-α-methyl[1,1′-biphenyl]-4-acetic acid) (Eriksen et al., J. Clin. Invest. 112:440 (2003)), L-685,458 ((5S)-(tert-Butoxycarbonylamino)-6-phenyl-(4R)-hydroxy-(2R)-benzylhexanoyl)-L-leucy-L-phenylalaninamide) (Shearman et al., Biochemistry 39:8698 (2000)), JLK 6 (7-Amino-4-chloro-3-methoxy-1H-2-benzopyran) (Petit et al., Nat. Cell. Biol. 3:507 (2001)), MRK 560 (N-[cis-4-[(4-Chlorophenyl)sulfonyl]-4-(2,5-difluorophenyl)cyclohexyl]-1,1,1-trifluoromethanesulfonamide) (Best et al., J. Pharm. Exp. Ther. 317:786 (2006)), PF 3084014 hydrobromide ((2S)-2-[[(2S)-6,8-Difluoro-1,2,3,4-tetrahydro-2-naphthalenyl]amino]-N-[1-[2-[(2,2-dimethylpropyl)amino]-1,1-dimethylethyl]-1H-imidazol-4-yl]pentanamide dihydrobromide) (Lanz et al., J. Pharmacol. Exp. Ther. 334:269 (2010)), or derivatives thereof.

In certain embodiments, the term “DAPT” refers to one example of a γ-secretase inhibitor that inhibits NOTCH, which is described as a dipeptidic γ-secretase-specific inhibitor otherwise known as N-[(3,5-Difluorophenyl)acetyl]-L-alanyl-2-phenyl]glycine-1,1-dimethyl ethyl ester; LY-374973, N—[N-(3,5-Difluorophenacetyl)-L-alanyl]-S-phenylglycine t-butyl ester; N—[N-(3,5-difluorophenacetyl)-L-alanyl]-S-phenylglycine t-butyl ester; with a chemical formula of C₂₃H₂₆F₂N₂O₄. One example of a DAPT derivative is DAP-BpB (N—[N-(3,5-difluorophenacetyl)-L-alanyl]-(S)-phenylglycine-4-(4-(8-bioti-namido)octylamino)benzoyl)benzyl)methylamide), a photoactivable DAPT derivative. In certain embodiments, DAPT has the following structure:

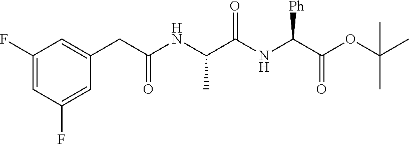 [SMILES: C[C@H](NC(=O)Cc1cc(F)cc(F)c1)C(=O)N[C@H](C(=O)OC(C)(C)C)c1ccccc1]

In certain embodiments, the at least one Notch inhibitor is selected from DAPT, BMS 299897, Compound E, Compound W, DBZ, L-685,458, PF 3084014 hydrobromide, derivatives thereof, and combinations thereof.

The inventors discovered that the initial contact or exposure of the cells with the at least one Notch inhibitor can impact the purity of the cortical neurons (e.g., cortical excitatory neurons), e.g., to obtain a population of pure and synchronized cortical neurons (e.g., cortical excitatory neurons). In addition, these cortical neurons (e.g., cortical excitatory neurons) can be maintained in long-term culture.

In certain embodiments, the initial contact or exposure of the cells (e.g., cortical progenitors) with or to the at least one Notch inhibitor is at least about 10 days, at least about 15 days, or at least about 20 days from the initial contact or exposure of the cells with or to the at least one SMAD inhibitor. In certain embodiments, the initial contact or exposure of the cells with or to the at least one Notch inhibitor is no later than about 15 days, no later than about 20 days, or no later than about 25 days from initial exposure of the stem cells to the at least one SMAD inhibitor. In certain embodiments, the initial contact or exposure of the cells with or to the at least one Notch inhibitor is between about 10 days and about 25 days, between about 10 days and about 20 days, between about 10 days and about 15 days, or between about 15 days and about 20 days from the initial contact of the cells with the at least one SMAD inhibitor. In certain embodiments, the initial contact of the cells to the at least one Notch inhibitor is about 20 days from the initial contact of the cells with the at least one SMAD inhibitor.

In certain embodiments, the cells (e.g., cortical progenitors) are exposed to or contacted with the at least one Notch inhibitor for at least about 1 day, at least about 5 days, at least about 10 days, at least about 15 days, at least about 20 days, at least about 25 days, or at least about 30 days. In certain embodiments, the cells (e.g., cortical progenitors) are exposed to or contacted with the at least one Notch inhibitor for at least about 1 day. In certain embodiments, the cells are contacted with or exposed to the at least one Notch inhibitor for up to about 5 days, about 10 days, up to about 15 days, up to about 20 days, up to about 25 days, or up to about 30 days. In certain embodiments, the cells are contacted with or exposed to the at least one Notch inhibitor for up to about 10 days. In certain embodiments, the cells are contacted with or exposed to the at least one Notch inhibitor for between about 1 day and about 30 days, between about 1 day and about 20 days, between about 1 day and about 15 days, between about 1 day and about 10 days, between about 1 day and about 5 days, between about 10 days and about 15 days, between about 10 days and about 20 days, between about 20 days and about 30 days, between about 10 days and about 30 days, or between about 15 days and about 20 days. In certain embodiments, the cells are contacted with or exposed to the at least one Notch inhibitor for between about 1 day and about 10 days. In certain embodiments, the cells are contacted with or exposed to the at least one Notch inhibitor for about 10 days. In certain embodiments, the stem cells are contacted with or exposed to the at least one Notch inhibitor from about day 20 through about day 30, where day 0 is the day when the initial exposure of the cells with the at least one SMAD inhibitor. In certain embodiments, the at least one Notch inhibitor is exposed to the cells daily, every other day, or every about 5 days. In certain embodiments, the at least one Notch inhibitor is added daily, every other day, or about every 5 days to a cell culture medium comprising the cells (e.g., cortical progenitors) from about day 20 through about day 30. In certain embodiments, the at least one Notch inhibitor is added about every 5 days to a cell culture medium comprising the cells (e.g., cortical progenitors) from about day 20 through about day 30.

In certain embodiments, the concentration of the Notch inhibitor contacted with or exposed to the cells (e.g., cortical progenitors) is between about 1 μM and about 20 μM, between about 1 μM and about 10 μM, between about 1 μM and about 15 μM, between about 10 μM and about 15 μM, between about 5 μM and about 10 μM, between about 5 μM and about 15 μM, between about 5 μM and about 20 μM, or between about 15 μM and about 20 μM. In certain embodiments, the concentration of the Notch inhibitor contacted with or exposed to the cells is between about 5 μM and about 10 μM. In certain embodiments, the concentration of the Notch inhibitor contacted with or exposed to the cells is about 10 μM. In certain embodiments, the Notch inhibitor comprises DAPT or a derivative thereof. In certain embodiments, the Notch inhibitor comprises DAPT.

In certain embodiments, before the initial contact of the cells with the at least one Notch inhibitor, the cells are dissociated to be re-plated.

### 5.2.3. Cell Culture Media

In certain embodiments, the above-described inhibitors are added to a cell culture medium comprising the cells. Suitable cell culture media include, but are not limited to, Knockout® Serum Replacement (“KSR”) medium, Neurobasal® medium (NB), N2 medium, B-27 medium, and Essential 8 ®/Essential 6 ® (“E8/E6”) medium, and combinations thereof. KSR medium, NB medium, N2 medium, B-27 medium, and E8/E6 medium are commercially available. KSR medium is a defined, serum-free formulation optimized to grow and maintain undifferentiated hESCs in culture.

In certain embodiments, the cell culture medium is an E8/E6 medium. E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods 2011 May; 8(5):424-9, which is incorporated by reference in its entirety. One example E8/E6 medium is disclosed in WO15/077648, which is incorporated by reference in its entirety. In certain embodiments, an E8/E6 cell culture medium comprises DMEM/F12, ascorbic acid, selenium, insulin, NaHCO₃, transferrin, FGF2 and TGFβ. The E8/E6 medium differs from a KSR medium in that E8/E6 medium does not include an active BMP or Wnt ingredient. Thus, in certain embodiments, when an E8/E6 medium is used to culture the presently disclosed population of stem cells to differentiate into a population of cortical progenitors, at least one inhibitor of SMAD signaling (e.g., those inhibiting BMP) is not required to be added to the E8/E6 medium. In certain embodiments, the cell culture medium for differentiation of stem cells to cortical progenitors (e.g., as described in Section 5.2.1) is an E8/E6 medium comprising at least one SMAD inhibitor (e.g., SB431542, optionally LDN19318). In certain embodiments, the cell culture medium for differentiation of stem cells to cortical progenitors from stem cells (e.g., as described in Section 5.2.1) is an E8/E6 medium comprising at least one SMAD inhibitor (e.g., SB431542, optionally LDN19318), and at least one Wnt inhibitor. In certain embodiments, the cell culture medium from day 0 to day 2 is an E8/E6 medium comprising at least one SMAD inhibitor (e.g., SB431542, optionally LDN19318), and at least one Wnt inhibitor. In certain embodiments, the cell culture medium from day 3 to day 9 is an E8/E6 medium comprising at least one SMAD inhibitor (e.g., SB431542, optionally LDN19318).

In certain embodiments, the cell culture medium for differentiation of stem cells to cortical progenitors (e.g., as described in Section 5.2.1) is a N2/B27 medium. In certain embodiments, the cell culture medium from day 10 to day 20 is a N2 medium supplemented with B27.

In certain embodiments, the cell culture medium for differentiation of cortical progenitors to cortical excitatory neurons (e.g., as described in Section 5.2.2) is a NB medium comprising at least one Notch inhibitor (e.g., DAPT). In certain embodiments, the cell culture medium from day 20 to day 29 is a N2 medium comprising at least one Notch inhibitor (e.g., DAPT). In certain embodiments, the N2 medium is further supplemented with B27, L-glutamine, and Y-27632.

In certain embodiments, the cortical excitatory neurons (e.g., obtained according to the methods described in Section 5.2.2) are cultured in a maturation medium. In certain embodiments, the maturation medium is a NB medium. In certain embodiments, the NB medium comprises one or more molecules selected from L-glutamine, brain-derived neurotrophic factor (BDNF), glial cell-derived neurotrophic factor (GDNF), Cyclic adenosine monophosphate (cAMP), and ascorbic acid (AA).

In certain embodiments, the cortical excitatory neurons can be maintained long term in the maturation medium. In certain embodiments, the cortical excitatory neurons can be maintained in the maturation medium for at least about 10 days, at least about 50, at least about 10 weeks, at least about 15 weeks, at least about 20 weeks, at least about 25 weeks, at least about 30 weeks, or at least about 40 weeks. In certain embodiments, the cortical excitatory neurons can be maintained in the maturation medium for about 10 weeks. In certain embodiments, the cell culture medium is a KSR medium. The components of a KSR medium are disclosed in WO2011/149762. In certain embodiments, a KSR medium comprises Knockout DMEM, Knockout Serum Replacement, L-Glutamine, Pen/Strep, MEM, and 13-mercaptoethanol. In certain embodiments, 1 liter of KSR medium comprises 820 mL of Knockout DMEM, 150 mL of Knockout Serum Replacement, 10 mL of 200 mM L-Glutamine, 10 mL of Pen/Strep, 10 mL of 10 mM MEM, and 55 μM of 13-mercaptoethanol.

### 5.3. Cell Populations and Compositions

The present disclosure provides a cell population of in vitro differentiated cells, wherein at least about 50% (e.g., at least about 55%, at least about 60%, at least about 65%, at least about 70%, at least about 75%, at least about 80%, at least about 85%, at least about 90%, at least about 95%, or at least about 99%) of the differentiated cells express at least one cortical excitatory neuron marker. In certain embodiments, at least about 75% of the differentiated cells express the at least one cortical excitatory neuron marker. In certain embodiments, at least about 95% of the differentiated cells express the at least one cortical excitatory neuron marker.

The presently disclosed cortical excitatory neuron cell population can be maintained in vitro long-term. In certain embodiments, the presently disclosed cortical excitatory neuron cell population can be maintained in vitro for at least about 10 days, at least about 50, at least about 10 weeks, at least about 15 weeks, at least about 20 weeks, at least about 25 weeks, at least about 30 weeks, or at least about 40 weeks.

Non-limiting examples of cortical excitatory neuron markers include TBR1, MAP2, CTIP2, FOXG1, DCX, TUBB3, FOXP2, vGlut1/2, and TLE4.

The presently disclosure also provides compositions comprising such cortical excitatory neuron cell populations. In certain embodiments, the in vitro differentiated cells are obtained by the differentiation methods described herewith, for example, in Section 5.2.

In certain embodiments, less than about 30% (e.g., less than about 25%, less than about 20%, less than about 15%, less than about 10%, less than about 5%, less than about 4%, less than about 3%, less than about 2%, less than about 1%, less than about 0.5%, or less than about 0.1%) of the differentiated cells express at least one marker selected from KI67, CTIP2 (Chicken Ovalbumin Upstream Promoter Transcription Factor Interacting Protein 2), SATB2 (Special AT-Rich Sequence-Binding Protein 2), and combinations thereof.

In certain embodiments, the cells are comprised in a composition that further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition comprises from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ in vitro differentiated cortical excitatory neurons.

In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier. The compositions can be used for preventing and/or treating a neurodegenerative disease, a neurodevelopmental disease, and/or a neuropsychiatric disorder. Non-limiting examples of neurodegenerative diseases, neurodevelopmental diseases, and/or neuropsychiatric disorders include Alzheimer's disease, Frontotemporal dementia, Parkinson's disease, schizophrenia, Autism, Depression, Intellectual disabilities, Amyotrophic lateral sclerosis, and Stroke.

The presently disclosed subject matter also provides a device comprising the differentiated cortical excitatory neurons or the composition comprising thereof, as disclosed herein. Non-limiting examples of devices include syringes, fine glass tubes, stereotactic needles and cannulas.

### 5.4. Method of Treating Neurodegenerative Disorders

The cell populations and compositions disclosed herein (e.g., those disclosed in Section 5.3) can be used for preventing and/or treating a neurodegenerative disease, a neurodevelopmental disorder, and/or a neuropsychiatric disorder. The presently disclosed subject matter provides for methods of preventing and/or treating a neurodegenerative disorder, a neurodevelopmental disorder, and/or a neuropsychiatric disorder. In certain embodiments, the method comprises administering the presently disclosed stem-cell-derived cortical excitatory neurons or a composition comprising thereof to a subject suffering from a neurodegenerative disorder, a neurodevelopmental disorder, and/or a neuropsychiatric disorder. In certain embodiments, the composition is a pharmaceutical composition further comprising a pharmaceutically acceptable carrier.

Non-limiting examples of a neurodegenerative disorders include Alzheimer's disease, Frontotemporal dementia, Parkinson's disease, schizophrenia, Autism, Depression, Intellectual disabilities, Amyotrophic lateral sclerosis, and Stroke.

In certain embodiments, the neurodegenerative disease, a neurodevelopmental disorder, and/or a neuropsychiatric disorder is Parkinson's disease. Primary motor signs of Parkinson's disease include, for example, but not limited to, tremor of the hands, arms, legs, jaw and face, bradykinesia or slowness of movement, rigidity or stiffness of the limbs and trunk and postural instability or impaired balance and coordination.

In certain embodiments, the Parkinson's disease refers to diseases that are linked to an insufficiency of dopamine in the basal ganglia, which is a part of the brain that controls movement. Symptoms include tremor, bradykinesia (extreme slowness of movement), flexed posture, postural instability, and rigidity. Non-limiting examples of parkinsonism diseases include corticobasal degeneration, Lewy body dementia, multiple systematrophy, and progressive supranuclear palsy.

The cells or compositions can be administered or provided systemically or directly to a subject for treating or preventing a neurodegenerative disorder, a neurodevelopmental disorder, and/or a neuropsychiatric disorder. In certain embodiments, the cells or compositions are directly injected into an organ of interest (e.g., the central nervous system (CNS)). In certain embodiments, the cells or compositions are directly injected into the cerebral cortex.

The cells or compositions can be administered in any physiologically acceptable vehicle. The cells or compositions can be administered via localized intracranial injection, orthotopic (OT) injection, systemic injection, intravenous injection, or parenteral administration. In certain embodiments, the cells or compositions are administered to a subject suffering from a neurodegenerative disorder via intracranial injection, e.g., localized intracranial injection into the CNS.

The cells or compositions can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived cortical excitatory neurons, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived precursors. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the cells is the quantity of cells necessary to achieve an optimal effect. An optimal effect includes, but is not limited to, repopulation of CNS regions of a subject suffering from a neurodegenerative disorder, and/or improved function of the subject's CNS.

In certain embodiments, the composition comprises an effective amount of the stem-cell-derived cortical excitatory neurons. As used herein, the term “effective amount” or “therapeutically effective amount” refers to an amount sufficient to affect a beneficial or desired clinical result upon treatment. An effective amount can be administered to a subject in at least one doses. In terms of treatment, an effective amount is an amount that is sufficient to palliate, ameliorate, stabilize, reverse or slow the progression of the neurodegenerative disorder or pituitary disorder, or otherwise reduce the pathological consequences of the neurodegenerative disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the cells is an amount that is sufficient to repopulate CNS regions of a subject suffering from a neurodegenerative disorder, a neurodevelopmental disorder, and/or a neuropsychiatric disorder. In certain embodiments, an effective amount of the cells is an amount that is sufficient to improve the function of the CNS of a subject suffering from a neurodegenerative disorder, a neurodevelopmental disorder, and/or a neuropsychiatric disorder, e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the cells are administered to a subject. In certain embodiments, from about 1×10⁵ to about 1×10⁷ of the cells are administered to a subject suffering from a neurodegenerative disorder. In certain embodiments, from about 1×10⁶ to about 1×10⁷ of the cells are administered to a subject suffering from a neurodegenerative disorder. In certain embodiments, from about 1×10⁶ to about 4×10⁶ of the cells are administered to a subject suffering from a neurodegenerative disorder. The precise determination of what would be considered an effective dose may be based on factors individual to each subject, including their size, age, sex, weight, and condition of the particular subject. Dosages can be readily ascertained by those skilled in the art from this disclosure and the knowledge in the art.

### 5.5. Kits

The presently disclosed subject matter provides kits for inducing differentiation of stem cells to cortical excitatory neurons thereof. In certain embodiments, the kit comprises (a) at least one inhibitor of SMAD signaling, (b) at least one inhibitor of Wnt signaling, and (c) at least one inhibitor of Notch signaling. In certain embodiments, the kit further comprises (d) instructions for inducing differentiation of the stem cells into a population of differentiated cells that express at least one cortical excitatory neuron marker.

In certain embodiments, the instructions comprise contacting the stem cells with the inhibitors in a specific sequence.

In certain embodiments, the instructions comprise contacting the stem cells with the inhibitors in compliance with the methods described in Section 5.2.

In certain embodiments, the present disclosure provides kits comprising an effective amount of a cell population or a composition disclosed herein in unit dosage form. In certain embodiments, the kit comprises a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit comprises instructions for administering the cell population or composition to a subject suffering from a neurodegenerative disorder, a neurodevelopmental disorder, and/or a neuropsychiatric disorder. The instructions can comprise information about the use of the cells or composition for treating and/or preventing a neurodegenerative disorder, a neurodevelopmental disorder, and/or a neuropsychiatric disorder. In certain embodiments, the instructions comprise at least one of the following: description of the therapeutic agent; dosage schedule and administration for treating and/or preventing a neurodegenerative disorder, a neurodevelopmental disorder, and/or a neuropsychiatric disorder, or symptoms thereof; precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 6. EXAMPLES

The presently disclosed subject matter will be better understood by reference to the following Example, which is provided as exemplary of the presently disclosed subject matter, and not by way of limitation.

### Example 1: Generation of Synchronized Pure Cortical Excitatory Neurons from hPSCs

The presently disclosed Example describes the directed differentiation of hPSCs toward a synchronized population of cortical excitatory neurons at very high purity in two steps:

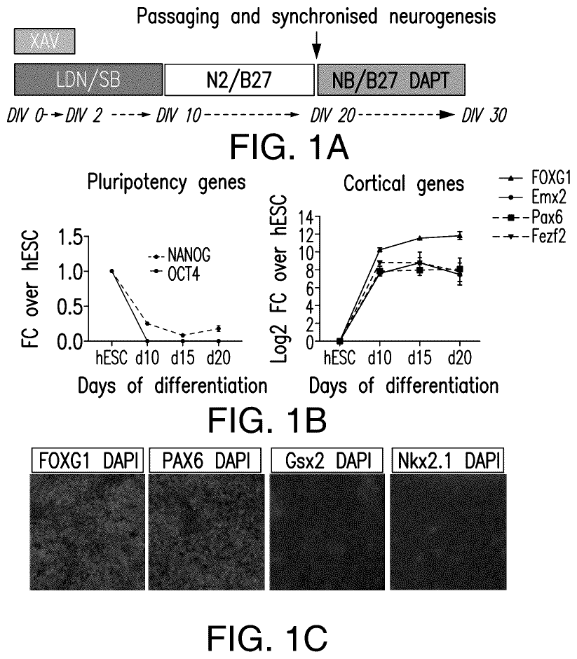

Step 1: hPSCs were robustly differentiated toward a homogeneous population of dorsal cortical progenitor cells expressing specific markers including FOXG1 and PAX6 (FIGS. 1A-1C);

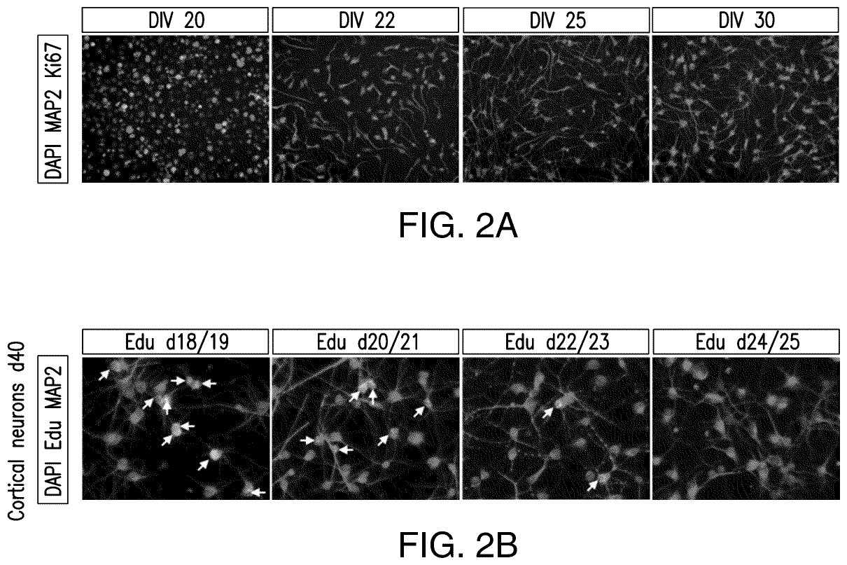

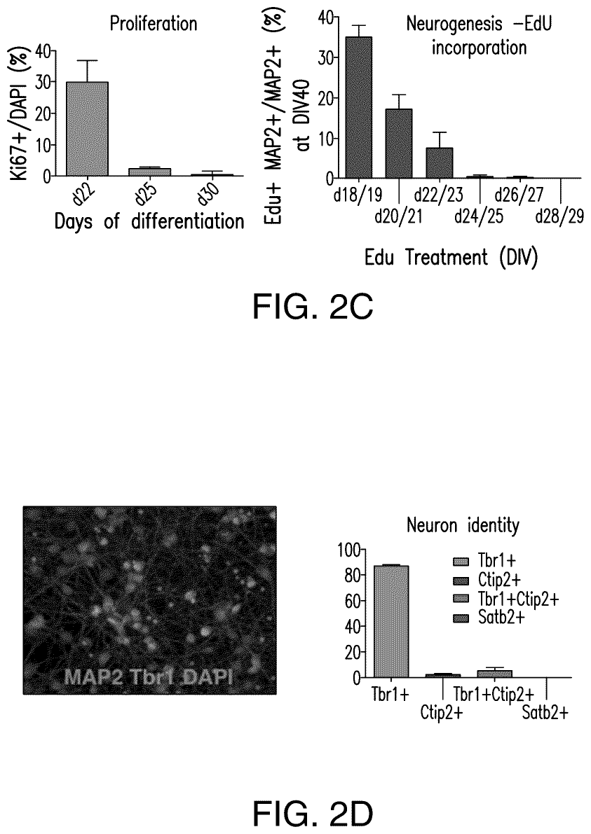

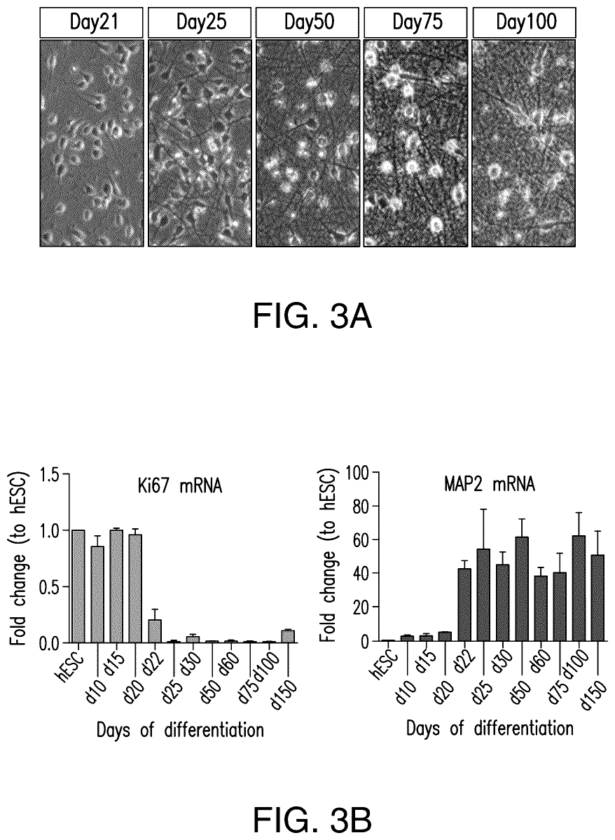

Step 2: Cortical progenitor cells were then passaged in conditions that promoted rapid cell cycle exit and induced the generation of a pure population of synchronized TBR1⁺ cortical excitatory neurons that can be maintain in long-term culture (FIGS. 2A-2D, 3A-3B)

Step 1 involved patterning of hPSCs toward an homogeneous population of cortical progenitor cells:
Day −1: hPSCs were dissociated at single-cells and plated at a density of 3×10⁵ cells/cm 2 on Matrigel-coated plates, and cells were cultured with E8 media containing 10 μM Y-27632.
Day 0 to Day 2: Cells were fed daily with E6 media supplemented with 10 μM SB431542, 100 nM LDN193189, and 2 μM XAV939.
Day 3 to Day 9: Cells were fed daily with E6 supplemented with 10 μM SB431542, and 100 nM LDN19318.
Day 10 to Day 20: Cells were fed daily with N2/B27 medium.

Step 2 involved passaging and synchronized neurogenesis:
Day 20-Day 29: Progenitor cells were dissociated into a single-cell suspension and plated at a density of 1-1.5×10⁵ cells/cm² onto plates coated with poly-L-ornithine, laminin, and fibronectin. Cells were cultured in NB medium supplemented with B27, L-glutamine, 10 uM Y-27632, and 10 uM DAPT, and half of the media of each plate was changed every 5 days until day 30.
Day 30-end point: Cells were maintained in NB supplemented with B27, L-glutamine, 20 μg/ml BDNF, 20 μg/ml GDNF, 200 μM dbcAMP, 200 μM Ascorbic Acid (AA). One half of the media was changed approximately every 4-5 days.

Step 1 of the presently disclosed methods robustly induced the differentiation of hPSCs toward a homogenous population of cortical progenitor cells. mRNA levels of the pluripotency-associated genes NANOG and OCT4, and of the telencephalic cortical markers FOXG1, Emx2, Pax6, and Fezf2 were determined. As shown in FIG. 1B, telencephalic cortical markers were progressively and robustly induced, pluripotency associated genes were efficiently reduced. Cortical-specific markers FOXG1 and PAX6 and ventral-telencephalic markers Gsx2 and Nkx2.1 were also evaluated. Immunofluorescence staining revealed homogeneous induction of cortical specific genes FOXG1 and PAX6 and minimal contamination of ventral telencephalic markers (FIG. 1C).

Step 2 of the presently disclosed methods rapidly depleted progenitor cells and synchronized neurogenesis. Ki67+ progenitor cells were rapidly depleted and generated a pure population of MAP2+ neurons by day 25 of differentiation (FIGS. 2A and 2C). 5-ethynyl-2′-deoxyuridine (EdU) birthdating confirmed the generation of roughly isochronic neurons (FIGS. 2B and 2C). Notably, the synchronized neurogenesis generated a highly pure population of neurons of a TBR1+identity (>80%) (FIG. 2D).

The presently disclosed methods were able to maintain the synchronized and pure hPSCs-derived cortical neurons at high viability in long-term cultures. Representative images of hPSCs-derived cortical neurons in long-term culture were shown in FIG. 3A. qRT-PCR analyses for KI67/MAP2 marker expression confirmed the maintenance of synchronicity and purity conditions in long-term cultures (FIG. 3B).

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the present disclosures of which are incorporated herein by reference in their entireties for all purposes

1. An in vitro method for inducing differentiation of stem cells, comprising: contacting the stem cells with at least one inhibitor of Small Mothers Against Decapentaplegic (SMAD) signaling, and at least one inhibitor of wingless (Wnt) signaling; and contacting the cells with at least one inhibitor of Notch signaling to obtain a cell population of differentiated cells, wherein at least about 50%, at least about 75%, or at least about 95% of the differentiated cells express at least one cortical excitatory neuron marker; wherein the initial contact of the cells with the at least one inhibitor of Notch signaling is at least about 10 days from the initial contact of the cells with the at least one inhibitor of SMAD signaling.

2. The method of claim 1, wherein the cells the initial contact of the cells with the at least one inhibitor of Notch signaling is about 20 days from the initial contact of the cells with the at least one inhibitor of SMAD signaling.

3. The method of claim 1, wherein the cells are contacted with the at least one inhibitor of Notch signaling for at least about 1 day.

4. The method of claim 1, wherein the cells are contacted with the at least one inhibitor of Notch signaling for up to about 20 days.

5. The method of claim 4, wherein the cells are contacted with the at least one inhibitor of Notch signaling for about 10 days.

6. The method of claim 1, wherein the cells are contacted with the at least one inhibitor of SMAD signaling for about 10 days.

7. The method of claim 1, wherein the cells are contacted with the at least one inhibitor of Wnt signaling for up to about 3 days.

8. The method of claim 1, wherein the at least one inhibitor of Notch signaling comprises a γ-secretase inhibitor.

9. The method of claim 8, wherein the γ-secretase inhibitor comprises DAPT, derivatives thereof, or mixtures thereof.

10. The method of claim 1, wherein the at least one inhibitor of SMAD signaling is selected from inhibitors of TGFβ/Activin-Nodal signaling, inhibitors of ALK5, inhibitors of bone morphogenetic protein (BMP) signaling, and combinations thereof.

11. (canceled)

12. The method of claim 10, wherein the at least one inhibitor of TGFβ/Activin-Nodal signaling comprises SB431542, or a derivative, or a mixture thereof.

13. (canceled)

14. The method of claim 10, wherein the at least one inhibitor of BMP signaling comprises LDN193189, Noggin, dorsomorphin, a derivative thereof, or a mixture thereof.

15. (canceled)

16. The method of claim 1, wherein the at least one inhibitor of Wnt signaling comprises a compound selected from the group consisting of XAV939, IWP2, DKK1, IWR1, IWP L6, Wnt-059, JW 55, derivatives thereof, and combinations thereof.

17. (canceled)

18. The method of claim 1, wherein the at least one cortical excitatory neuron marker is selected from TBR1 (T-Box Brain 1), MAP2 (Microtubule-Associated Protein 2), FOXG1, CTIP2, DCX, TUBB3, FOXP2, vGlut1/2, TLE4, and combinations thereof.

19. The method of claim 1, wherein the differentiated cells do no express at least one marker selected from KI67, CTIP2 (Chicken Ovalbumin Upstream Promoter Transcription Factor Interacting Protein 2), SATB2 (Special AT-Rich Sequence-Binding Protein 2), and combinations thereof.

20. The method of claim 1, wherein the stem cells are selected from pluripotent or multipotent stem cells; embryonic stem cells, induced pluripotent stem cells, and combinations thereof; human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells.

21. (canceled)

22. (canceled)

23. (canceled)

24. (canceled)

25. A cell population of in vitro differentiated cells, wherein the in vitro differentiated cells are obtained according to a method of claim 1.

26. (canceled)

27. (canceled)

28. (canceled)

29. A composition comprising the cell population of claim 25.

30. (canceled)

31. A kit for inducing differentiation of stem cells to cortical excitatory neurons, comprising:
(a) at least one inhibitor of SMAD signaling;
(b) at least one inhibitor of Wnt signaling;
(c) at least one inhibitor of Notch signaling; and
(d) instructions for inducing differentiation of the stem cells into a population of differentiated cells expressing at least one cortical excitatory neuron marker.

32. (canceled)

33. A method of preventing and/or treating a neurodegenerative disorder, a neurodevelopmental disorder, and/or a neuropsychiatric disorder in a subject, comprising administering to the subject an effective amount of the composition of claim 29, wherein the neurodegenerative disorder, a neurodevelopmental disorder, and/or a neuropsychiatric disorder is selected from Alzheimer's disease, Frontotemporal dementia, Parkinson's disease, schizophrenia, Autism, Depression, Intellectual disabilities, Amyotrophic lateral sclerosis, and Stroke.

34. (canceled)

35. (canceled)

36. (canceled)
